(19) United States
(12) Patent Application Publication
(10) Pub. No.: US 2023/0173262 A1
(43) Pub. Date: Jun. 8, 2023

(54) IMPLANTABLE MEDICAL DEVICE

(71) Applicant: Stephen T. Pyles, Ocala, FL (US)

(72) Inventor: Stephen T. Pyles, Ocala, FL (US)

(21) Appl. No.: 18/062,212

(22) Filed: Dec. 6, 2022

Related U.S. Application Data
(60) Provisional application No. 63/286,765, filed on Dec. 7, 2021.

(51) Int. Cl. A61N 1/05; A61N 1/375; A61N 1/378

(52) U.S. Cl. CPC A61N 1/0551; A61N 1/375; A61N 1/378

(57) ABSTRACT

An implantable medical device includes a power source configured to supply electrical impulses to a lead. A housing encloses the power source, and the housing includes a posterior surface, an anterior surface, and a circumferential edge extending between the posterior and anterior surfaces. The housing defines a storage area for excess slack of the lead.

### CROSS-REFERENCE TO RELATED APPLICATIONS

This application claims priority to, and the benefit of U.S. Provisional Patent Application No. 63/286,765, filed Dec. 7, 2021, which is hereby incorporated by reference in its entirety.

### BACKGROUND

Spinal cord stimulation is a medical therapy that is performed to alleviate chronic pain by stimulating the central nervous system. Typically, a distal end of a lead having electrical contacts is implanted in the epidural space of the spine, in close proximity to the spinal cord. A proximal end of the lead is connected to a stimulator, which includes a power source.

The stimulator is implanted into the buttock or lower back, and when activated, delivers electrical impulses to the electrical contacts on the distal end of the lead, for delivering the electrical impulses to the spinal cord or to a peripheral nerve. The electrical impulses activate pain inhibitory mechanisms to block the pain signal from reaching the brain, and thereby alleviate chronic pain such as for treating lower back pain.

### SUMMARY

In general terms, the present disclosure relates to storage of excess slack of a lead connected to an implantable medical device. In one possible configuration, an implantable medical device has a housing with a storage area for the excess slack of the lead. In another possible configuration, an accessory attaches to an implantable medical device, and the accessory provides a storage area for the excess slack of the lead. Various aspects are described in this disclosure, which include, but are not limited to, the following aspects.

In one aspect, an implantable medical device is described. The implantable medical device comprises: a power source configured to supply electrical impulses to a lead; and a housing encloses the power source, the housing having: a posterior surface; an anterior surface; and a circumferential edge extending between the posterior and anterior surfaces; and wherein the housing defines a storage area for excess slack of the lead.

In another aspect, an accessory for an implantable medical device is described. The accessory comprises: a posterior surface having a convex shape; an anterior surface having a cavity configured to attach the accessory to a spinal cord stimulator device, and to partially encapsulate the spinal cord stimulator device; a circumferential edge extending between the posterior and anterior surfaces; and at least one storage area shaped for storing excess slack of a lead when the lead is connected to the spinal cord stimulator device.

### DESCRIPTION OF THE FIGURES

The following drawing figures, which form a part of this application, are illustrative of the described technology and are not meant to limit the scope of the disclosure in any manner.

FIG. 1 is an isometric posterior view of an embodiment of an implantable medical device having a storage area for one or more leads having excess slack.

FIG. 2 is an isometric anterior view of the implantable medical device of FIG. 1.

FIG. 3 is a posterior view of the implantable medical device of FIG. 1.

FIG. 4 is an anterior view of the implantable medical device of FIG. 1.

FIG. 5 is a right side view of the implantable medical device of FIG. 1.

FIG. 6 is a left side view of the implantable medical device of FIG. 1.

FIG. 7 is a top view of the implantable medical device of FIG. 1.

FIG. 8 is a bottom view of the implantable medical device of FIG. 1.

FIG. 9 is an isometric anterior view of the implantable medical device of FIG. 1 with the excess slack of the one or more leads stored in the storage area.

FIG. 10 is an isometric posterior view of another embodiment of the implantable medical device having a storage area for one or more leads having excess slack.

FIG. 11 is an isometric anterior view of the implantable medical device of FIG. 10.

FIG. 12 is a posterior view of the implantable medical device of FIG. 10.

FIG. 13 is an anterior view of the implantable medical device of FIG. 10.

FIG. 14 is a right side view of the implantable medical device of FIG. 10.

FIG. 15 is a left side view of the implantable medical device of FIG. 10.

FIG. 16 is a top view of the implantable medical device of FIG. 10.

FIG. 17 is a bottom view of the implantable medical device of FIG. 10.

FIG. 18 a cross-sectional view of the implantable medical device of FIG. 10.

FIG. 19 is an isometric posterior view of another embodiment of the implantable medical device having a storage area for one or more leads having excess slack.

FIG. 20 is an isometric anterior view of the implantable medical device of FIG. 19.

FIG. 21 is a posterior view of the implantable medical device of FIG. 19.

FIG. 22 is an anterior view of the implantable medical device of FIG. 19.

FIG. 23 is a cross-sectional view of the implantable medical device of FIG. 19 showing the excess slack of the one or more leads stored in the storage area.

FIG. 24 is an isometric posterior view of an embodiment of an accessory for an implantable medical device, the accessory having a storage area for excess lead slack.

FIG. 25 is an isometric anterior view of the accessory of FIG. 24.

FIG. 26 is a posterior view of the accessory of FIG. 24.

FIG. 27 is an anterior view of the accessory of FIG. 24.

FIG. 28 is a right side view of the accessory of FIG. 24.

FIG. 29 is a left side view of the accessory of FIG. 24.

FIG. 30 is a top view of the accessory of FIG. 24.

FIG. 31 is a bottom view of the accessory of FIG. 24.

FIG. 32 is a cross-sectional view of the accessory of FIG. 24.

FIG. 33 is an isometric posterior view of another embodiment of the accessory for an implantable medical device, the accessory having a storage area for excess lead slack.

FIG. 34 is an isometric anterior view of the accessory of FIG. 33.

FIG. 35 is a posterior view of the accessory of FIG. 33.

FIG. 36 is an anterior view of the accessory of FIG. 33.

FIG. 37 is a right side view of the accessory of FIG. 33.

FIG. 38 is a left side view of the accessory of FIG. 33.

FIG. 39 is a top view of the accessory of FIG. 33.

FIG. 40 is a bottom view of the accessory of FIG. 33.

FIG. 41 is a cross-sectional view of the accessory of FIG. 33.

FIG. 42 schematically illustrates components of the implantable medical device in accordance with any one of the embodiments shown in FIGS. 1-41.

### DETAILED DESCRIPTION

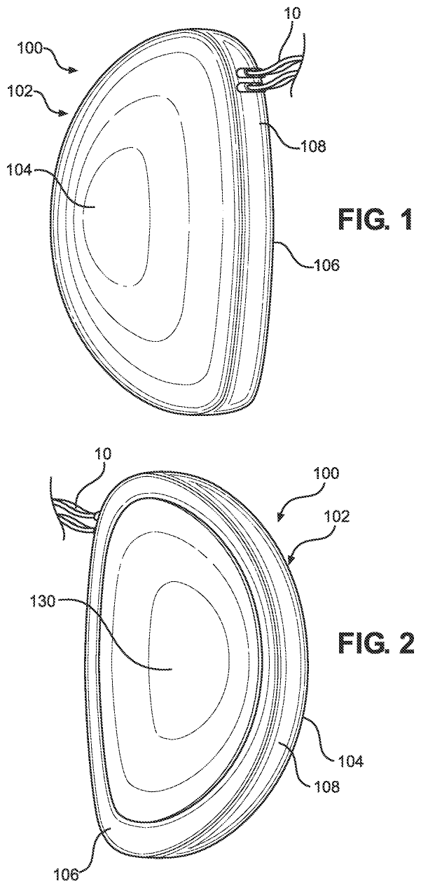

FIGS. 1 and 2 are isometric posterior and anterior views, respectively, of an implantable medical device 100. As shown in FIGS. 1 and 2, one or more leads 10 are connected to the implantable medical device 100. As will be described in more detail, the implantable medical device 100 has a storage area for storing excess slack from the one or more leads 10 when the implantable medical device 100 and the one or more leads 10 are implanted.

The implantable medical device 100 includes a housing 102 having a posterior surface 104, an anterior surface 106, and a circumferential edge 108 extending between the posterior and anterior surfaces 104, 106. As used herein, “posterior” refers to the back of the human body and “anterior” refers to the front of the human body. Accordingly, the posterior surface 104 is directed toward the back of the body, while the anterior surface 106 is directed toward the front of the body when the implantable medical device 100 is implanted.

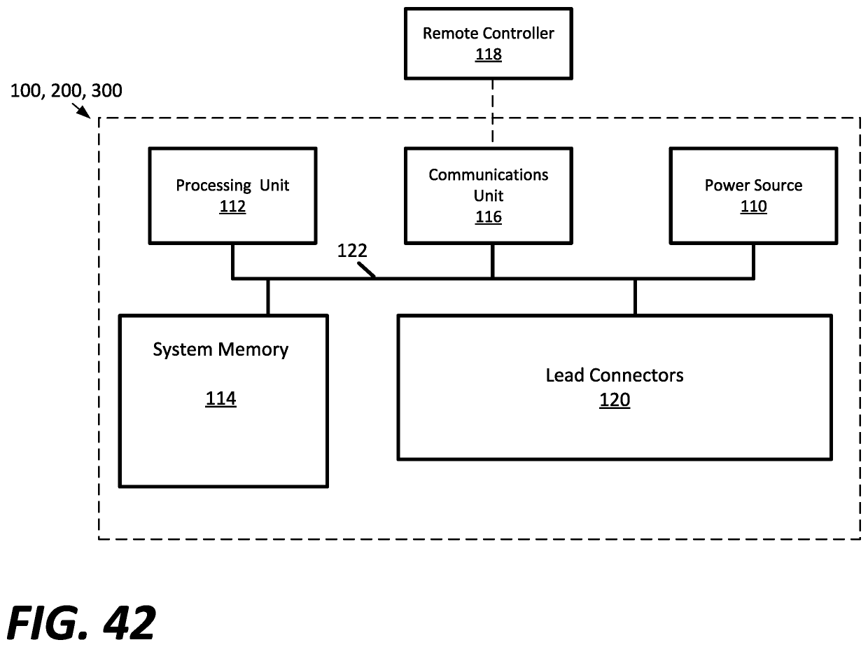

In a preferred embodiment, the implantable medical device 100 is a spinal cord stimulator device that has a power source 110 (see FIG. 42) programmed to generate electrical impulses for transmission through the one or more leads 10. In some examples, the one or more leads 10 are percutaneous leads. In other examples, the one or more leads 10 are paddle leads. It is contemplated that in alternative embodiments, the implantable medical device 100 may be a different kind of medical device that is implantable into a body of a patient.

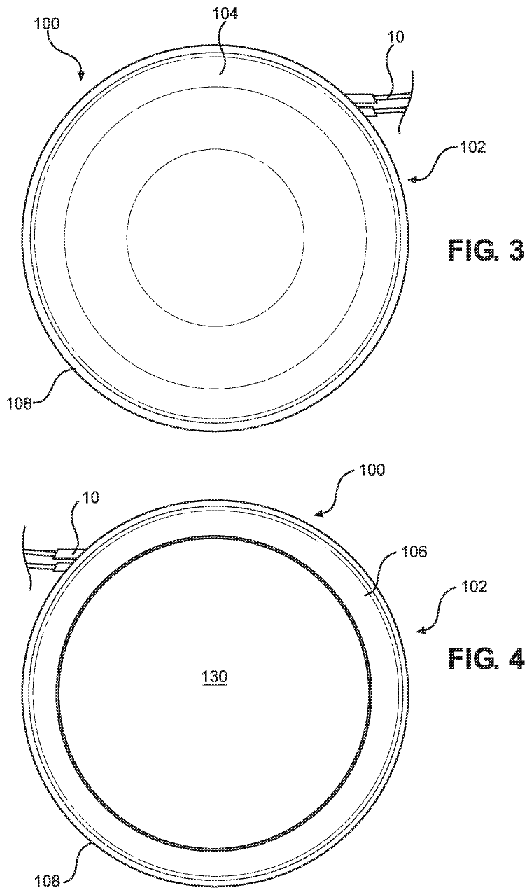

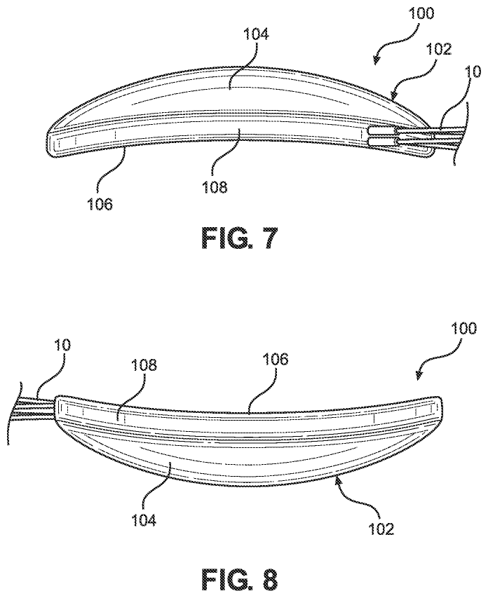

FIGS. 3-8 are anterior, posterior, right side, left side, top, and bottom views, respectively, of the implantable medical device 100. As shown in FIGS. 1-8, the posterior surface 104, the anterior surface 106, and the circumferential edge 108 of the housing 102 form an enclosure for the power source 110. The housing 102 protects the power source 110 and other internal components from tissue and blood, as well as from impact with other objects when the implantable medical device 100 is implanted into a patient's body.

The housing 102 can be made of an inert and biocompatible material. For example, the housing 102 can be made of titanium or a titanium alloy, or similar materials.

Each lead of the one or more leads 10 extends from a proximal end to a distal end. Each lead has a standardized length. For example, the leads can have a standardized length of 50 cm or 70 cm. It is contemplated that the leads may have additional standardized lengths.

The figures illustrate an example in which four leads are connected to the implantable medical device 100. In some examples, fewer than four leads are connected to the implantable medical device 100 such as one, two, or three leads can be connected. In some further examples, more than four leads can be connected to the implantable medical device 100.

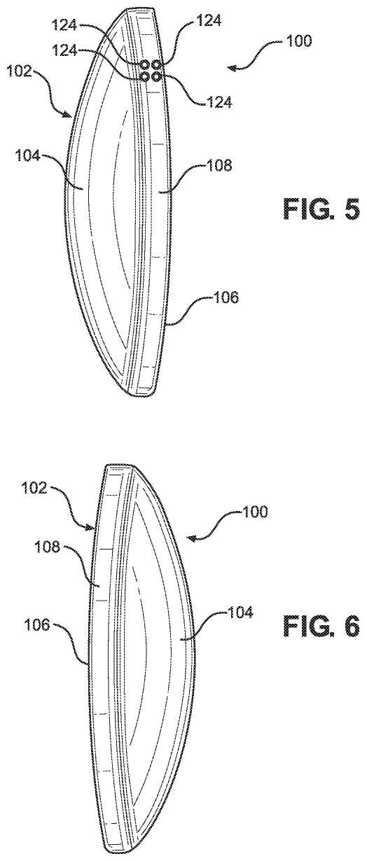

The figures further illustrate the housing 102 as having one or more openings 124 (see FIG. 5), which allow the leads 10 to enter housing 102 and attach to the power source 110. In the example provided in the figures, the housing 102 includes four openings, which allows up to four leads to attach to the power source 110. In alternative examples, the housing 102 can include more than four openings, or can include fewer than four openings. Also, while the one or more openings 124 are shown as positioned on the circumferential edge 108, it is contemplated that the one or more openings 124 can be positioned elsewhere on the housing 102.

The proximal end of each lead attaches to the implantable medical device 100, and the distal end of each lead has electrical contacts that release electrical impulses to an area of the human body. In examples where the implantable medical device 100 is a spinal cord stimulator device, a distal end of a lead is implanted in the epidural space of the spine, such that the electrical contacts on the distal end can release the electrical impulses to the spinal cord or to a peripheral nerve to mitigate pain in the back, abdomen, chest, and other areas of the body.

The location where the distal end of a lead is implanted may vary along the epidural space in the spine depending on a treatment plan selected for mitigating pain in the back, abdomen, chest, and other areas of the body. For example, a distal end of a lead can be implanted between the T11 and T12 vertebrae, between the T12 and L1 vertebrae, above the T11 vertebrae, or below the L1 vertebrae. In certain examples, a distal end of a lead can be implanted at the T7 vertebrae, or can be implanted between the T9 and T10 vertebrae. These locations are provided by way of illustrative example, and it is contemplated that the distal ends of the leads can be implanted in additional areas in the epidural space of the spinal column. Additionally, the distal ends of different leads can be implanted in different areas of the epidural space. The location where a distal end of a lead is implanted is referred to herein as a first body area.

The implantable medical device 100 is implanted in a second body area. In some examples, the second body area is the buttock or lower back. In some further examples, the second body area can be an underarm, on the chest wall, or under the scalp.

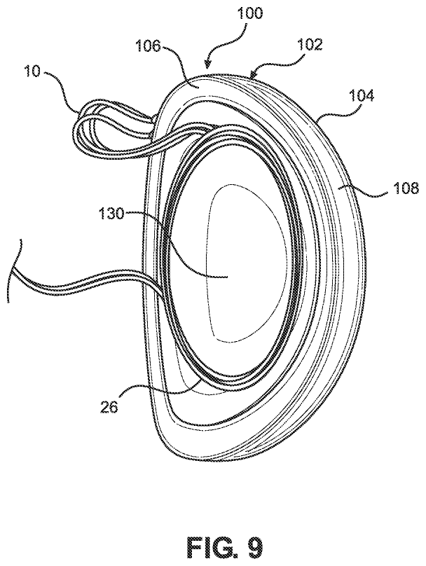

When a distal end of a lead from the one or more leads 10 is implanted in the first body area (e.g., the epidural space in the spine) and the implantable medical device 100 is implanted in the second body area (e.g., the buttock or lower back), one or more of the leads 10 can have excess slack 26 (see FIG. 9). As an illustrative example, a lead can have excess slack when the standardized length of the lead (e.g., 50 cm or 70 cm) is longer than the distance between the first and second body areas. Accordingly, the excess slack of a lead can be defined as an excess amount of a first length over a second length, in which the first length is defined as a distance between the proximal and distal ends of the lead (e.g., the standardized length of the lead), and the second length is defined as a distance between the first and second body areas.

The implantable medical device 100 is configured to store the excess slack 26 of one or more of the leads 10. Advantageously, storing the excess slack of the one or more leads 10 can reduce pain and discomfort that may be felt by a patient after the implantable medical device 100 and the one or more leads 10 have been implanted in the patient's body. Additionally, by storing the excess slack of the one or more leads 10 in a defined storage area can aid the removal or replacement of the one or more leads 10 of the implantable medical device 100.

FIG. 9 is an isometric anterior view of the implantable medical device 100 with the excess slack 26 of the one or more leads 10 stored in the storage area. Referring now to FIGS. 1-9, the housing 102 of the implantable medical device 100 is shaped to define a storage area for storing the excess slack 26 of the one or more leads 10. In this example, the storage area is a negative space 130 shaped by the anterior surface 106. For example, the anterior surface 106 has a concave shape that defines the negative space 130. In alternative examples, the negative space 130 can be formed as a cutout or a hollowed portion on the anterior surface 106.

In the example shown in FIGS. 1-9, the posterior surface 104 is convex. The convex shape of the posterior surface 104 provides a contour generally conforming to the profile of the second body area where the implantable medical device 100 is implanted. Advantageously, this minimizes the appearance of the implantable medical device 100 when implanted under the skin of a patient, and provides an advantage over conventional spinal cord stimulators, which can create a noticeable bulge with defined edges that are visible, even under clothing.

The convex shape of the posterior surface 104 can vary depending on the desired location for implanting the implantable medical device 100. For example, the convex shape of the posterior surface 104 can be varied to conform to the profile of a buttock, lower back, underarm, chest wall, scalp, and other suitable areas of the body for implanting the device.

As further shown in FIGS. 1-9, the circumferential edge 108 is circular. Advantageously, the convex shape of the posterior surface 104 and the circular profile of the circumferential edge 108 can help to improve the distribution forces that are applied to the implantable medical device 100 when implanted, and thereby improve the durability of the device. For example, when the implantable medical device 100 is implanted into the buttock of a patient, and the patient sits down, the convex shape of the posterior surface 104 and the circular profile of the circumferential edge 108 can help to improve the distribution of the patient's weight applied to the implantable medical device 100 as a result of the patient sitting down. In the example shown in the figures, the convex shape of the posterior surface 104 and the circular profile of the circumferential edge 108 provide the housing 102 with a dome-shape.

The convex shape of the posterior surface 104 and the circular profile of the circumferential edge 108 can result in an increased surface area and overall size of the implantable medical device 100 over conventional spinal cord stimulators. This is an unexpected result because conventional spinal cord stimulators are designed to be as small as possible.

As shown in FIG. 9, the excess slack 26 of the one or more leads 10 can be coiled for storage in the negative space 130 defined by the anterior surface 106. The shape of the housing 102 can completely cover the excess slack 26 of the one or more leads 10 such that excess slack 26 is not exposed under the skin surface of the patient. This can help to mitigate irritation and discomfort that may result from loose leads, and can minimize the appearance of the excess slack 26 of the one or more leads 10, which may bulge under the skin.

In some further examples, the excess slack 26 of the one or more leads 10 can be at least partially coiled around the circumferential edge 108 for storage. In such examples, the circular profile of the circumferential edge 108 can help facilitate coiling the excess slack 26 of the one or more leads 10 around the circumferential edge 108. In some examples, the circumferential edge 108 can include a track, groove, or channel for coiling the excess slack 26 of the one or more leads 10 around the circumferential edge 108.

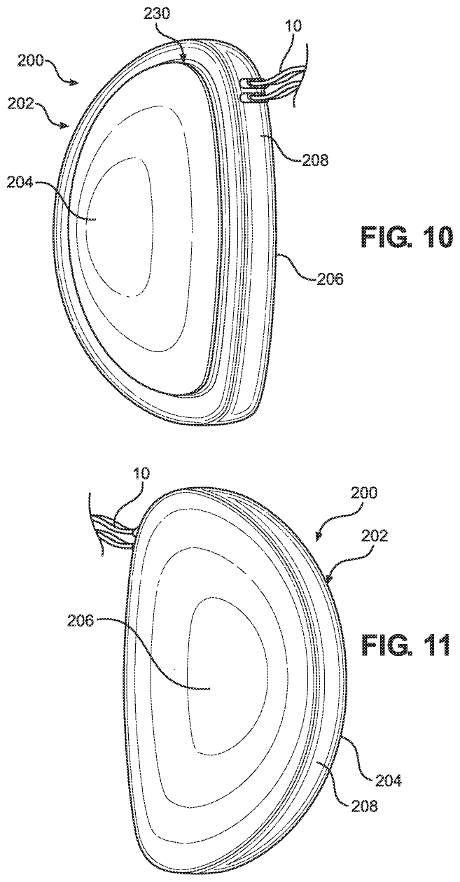

FIGS. 10 and 11 are isometric posterior and interior views, respectively, of an implantable medical device 200, in accordance with another embodiment of the present disclosure. As shown in FIGS. 10 and 11, one or more leads 10 are connected to the implantable medical device 200, which has a storage area for storing excess slack from the one or more leads 10 when the implantable medical device 200 and the one or more leads 10 are implanted.

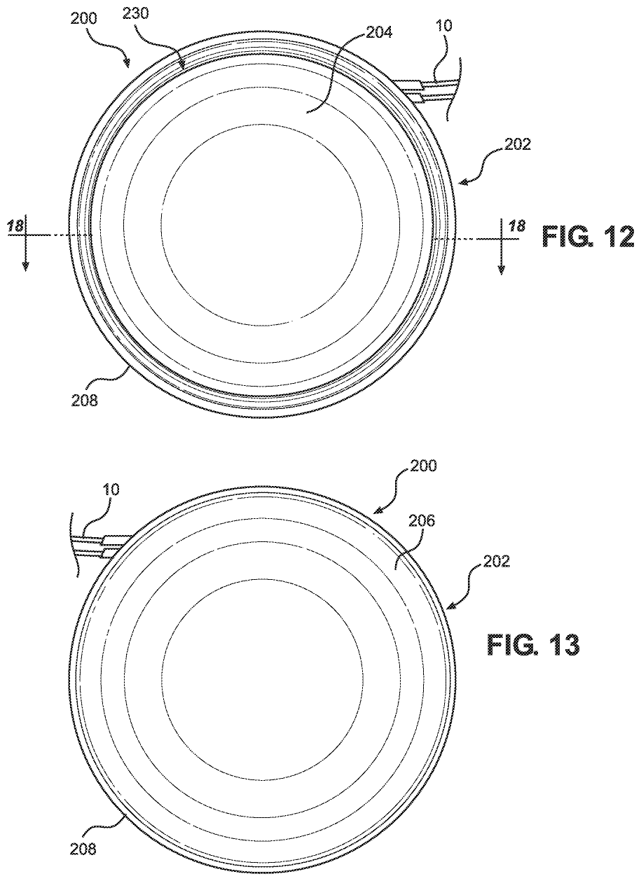

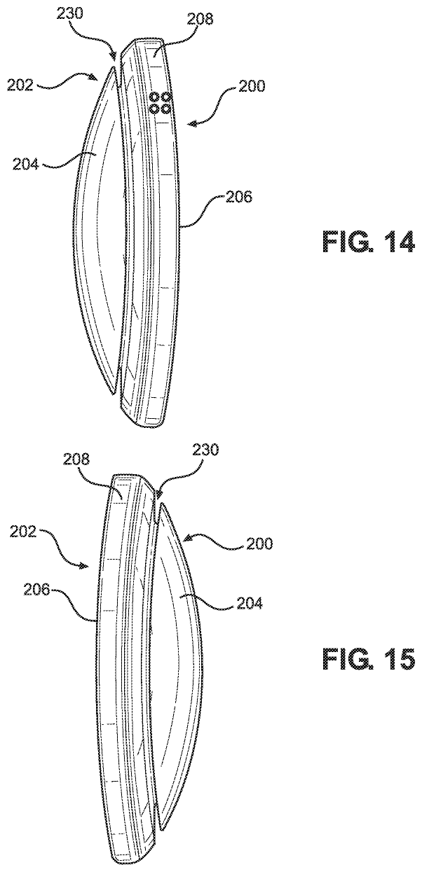

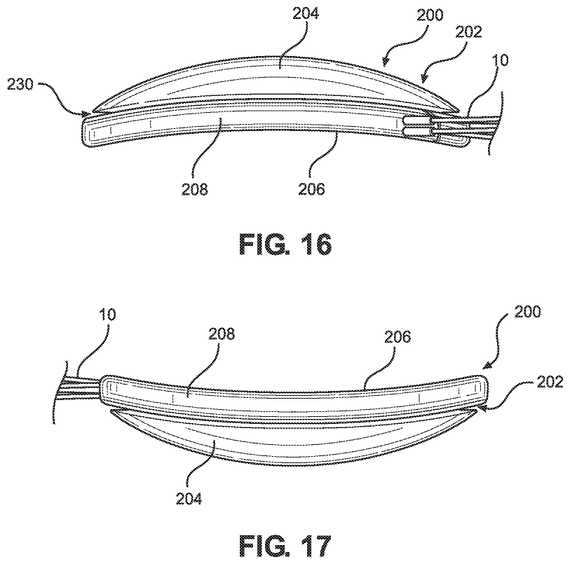

FIGS. 12-17 are posterior, anterior, right side, left side, top, and bottom views, respectively, of the implantable medical device 200. The implantable medical device 200 shares similar elements and features as the implantable medical device 200 described above with respect to FIG. 1-9. For example, the implantable medical device 200 includes a housing 202 having a posterior surface 204, an anterior surface 206, and a circumferential edge 208 extending between the posterior and anterior surfaces 204, 206. The housing 202 can be made of an inert and biocompatible material such as titanium or a titanium alloy, or similar materials.

As shown in FIGS. 10-17, the posterior surface 204 has a convex shape and the circumferential edge 208 has a circular profile. As described above, convex shape of the posterior surface 204 and the circular profile of the circumferential edge 208 can help to improve the distribution forces that are applied to the implantable medical device 200 when implanted, and thereby improve the durability of the implantable medical device 200.

The convex shape of the posterior surface 204 and the circular profile of the circumferential edge 208 can result in an increased surface area and overall size of the implantable medical device 200 over conventional spinal cord stimulators. This is an unexpected result because conventional spinal cord stimulators are designed to be as small as possible.

The housing 202 of the implantable medical device 200 defines a storage area for storing the excess slack of the one or more leads 10. In the example shown in FIGS. 10-17, the storage area is a track 230 defined between the circumferential edge 208 and the posterior surface 204 that allows the one or more leads 10 to be at least partially looped around the housing 202 for storage to reduce the slack of the leads. In alternative examples, the track 230 can be defined around the circumferential edge 208 of the housing 202.

In some examples, one or more of the leads 10 can be looped once, twice, or more around the housing 202 for storage to reduce the slack of the leads. In this example embodiment, another advantage of the circular profile of the circumferential edge 208 is that it facilitates wrapping, winding, or coiling the one or more leads 10 around the housing 202 for storage.

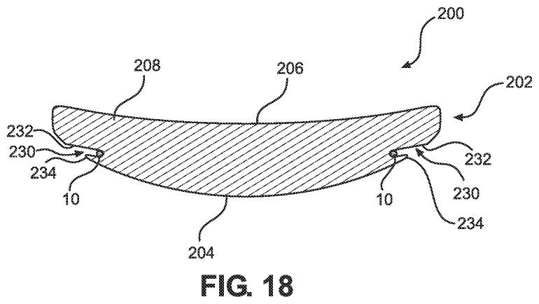

FIG. 18 a cross-sectional view of the implantable medical device 200 taken from the line 18-18 shown in FIG. 12. As shown in FIG. 18, the track 230 includes opposing edges 232, 234 that can catch one or more of the leads 10 for containing one or more of the leads 10 inside the track 230. In this example, the track 230 is channel or groove that is formed around the posterior surface 204. Advantageously, the track 230 can mitigate irritation and discomfort that may result from loose leads, and can minimize the appearance of the excess slack 26 from the one or more leads 10, which may bulge under the skin when left loose.

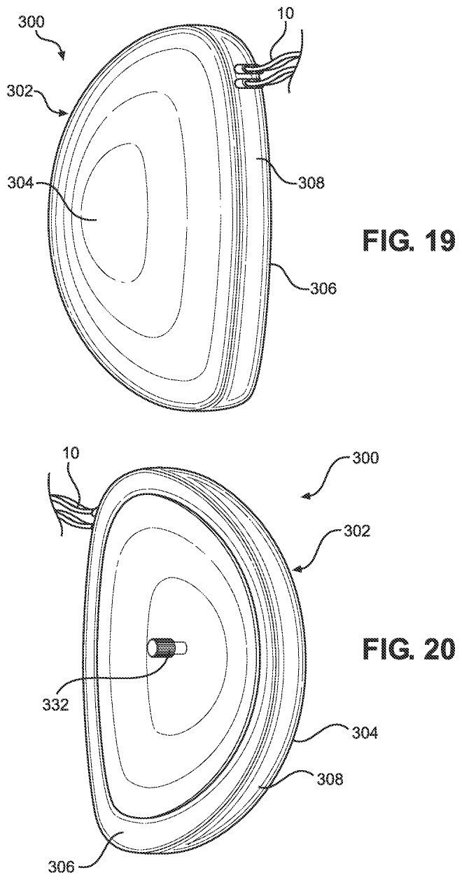

FIGS. 19 and 20 are isometric posterior and anterior views, respectively, of an implantable medical device 300, in accordance with another embodiment of the present disclosure. The implantable medical device 300 has a storage area for storing the excess slack 26 from the one or more leads 10 when the implantable medical device and leads are implanted.

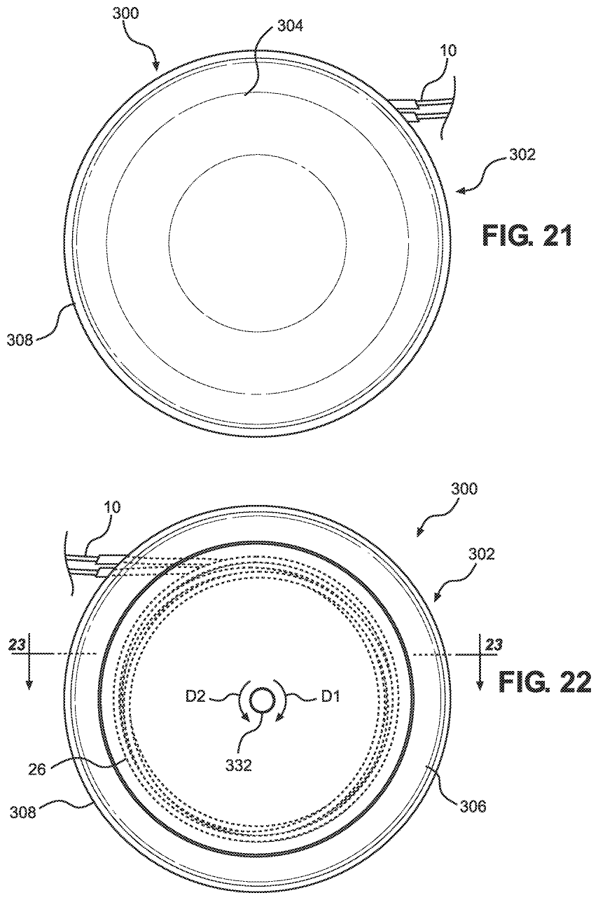

FIGS. 21 and 22 are posterior and anterior views, respectively, of the implantable medical device 300. As shown in FIGS. 19-22, the implantable medical device 300 shares similar elements and features as the implantable medical devices 100, 200 described above with respect to FIG. 1-18. For example, the implantable medical device 300 includes a housing 302 having a posterior surface 304, an anterior surface 306, and a circumferential edge 308 extending between the posterior and anterior surfaces 304, 306. The housing 302 can be made of an inert and biocompatible material such as titanium or a titanium alloy, or similar materials.

As shown in FIGS. 19-22, the posterior surface 304 has a convex shape and the circumferential edge 308 has a circular profile. As described above, convex shape of the posterior surface 304 and the circular profile of the circumferential edge 308 can help to improve the distribution forces that are applied to the implantable medical device 300 when implanted, and thereby improve the durability of the implantable medical device 300.

The convex shape of the posterior surface 304 and the circular profile of the circumferential edge 308 can result in the housing 302 having a dome-shape, which can increase surface area and overall size of the implantable medical device 300 over conventional spinal cord stimulators. As noted in the examples described above, this is an unexpected result because conventional spinal cord stimulators are designed to be as small as possible.

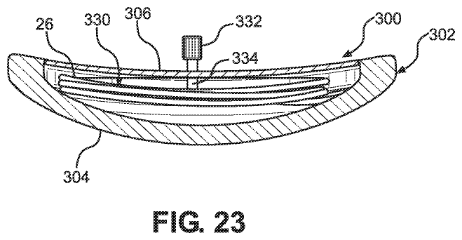

FIG. 23 is a cross-sectional view of the implantable medical device 300 taken from the line 23-23 shown in FIG. 22. As shown in FIGS. 22 and 23, the excess slack 26 of the one or more leads 10 can be stored in a storage area, which is a volume 330 defined inside the housing 302 of the implantable medical device 300. Advantageously, the housing 302 can protect the excess slack 26 of the one or more leads 10 from tissue and blood, as well as from impact with other objects when the implantable medical device 100 is implanted.

In this example embodiment, the housing 302 includes a crank 332 for winding or coiling the excess slack 26 of the one or more leads 10 for storage inside the volume 330 of the housing 302. The proximal ends of the one or more leads 10 can be attached or otherwise wrapped around a spool 334. The crank 332 can be rotated by the fingers of a physician when implanting the implantable medical device 300. The crank 332 can be rotated by a physician as necessary to adjust the excess slack 26 of the leads 10 outside of the volume 330.

As shown in FIG. 22, the crank 332 can be rotated in opposite directions to adjust the length of the one or more leads 10 outside of the volume 330 of the housing 302. For example, the crank 332 can be rotated in a first direction D1 (e.g., clockwise) to coil the one or more leads 10 around the spool 334, and thereby reduce the excess slack 26 outside of the volume 330. Also, the crank 332 can be rotated in a second direction D2 (e.g., counterclockwise) to uncoil the one or more leads 10, and thereby increase the excess slack 26 outside of the volume 330

Advantageously, the crank 332 can be used to adjust the excess slack 26 of the leads 10 outside of the volume 330 of the housing 302. Advantageously, this can mitigate irritation and discomfort that may result from loose leads under the patient's skin, and can minimize the appearance of the one or more leads 10, which may bulge under the skin.

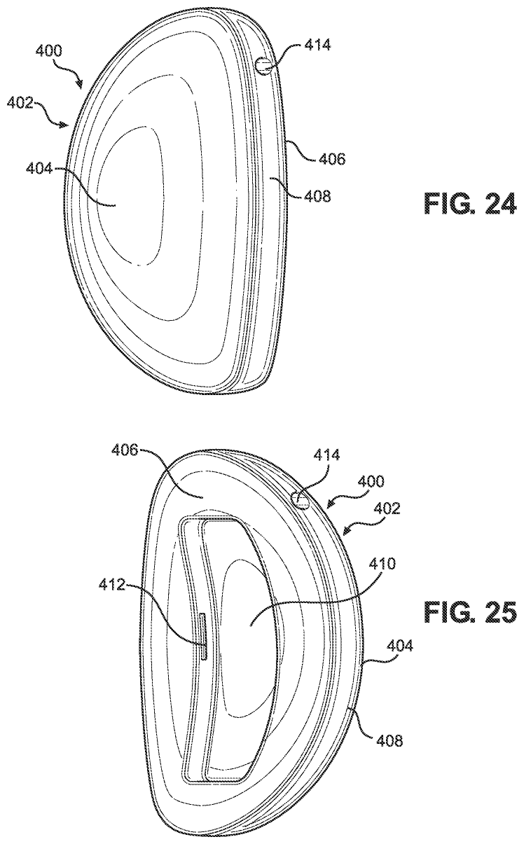

FIGS. 24 and 25 are isometric posterior and anterior views, respectively, of an accessory 400 for an implantable medical device. In a preferred embodiment, the accessory 400 is for a spinal cord stimulator device that has a power source 110 (see FIG. 42) programmed to generate electrical impulses for transmission through the one or more leads 10, and the accessory 400 provides a storage area for the excess slack 26 of the one or more leads 10.

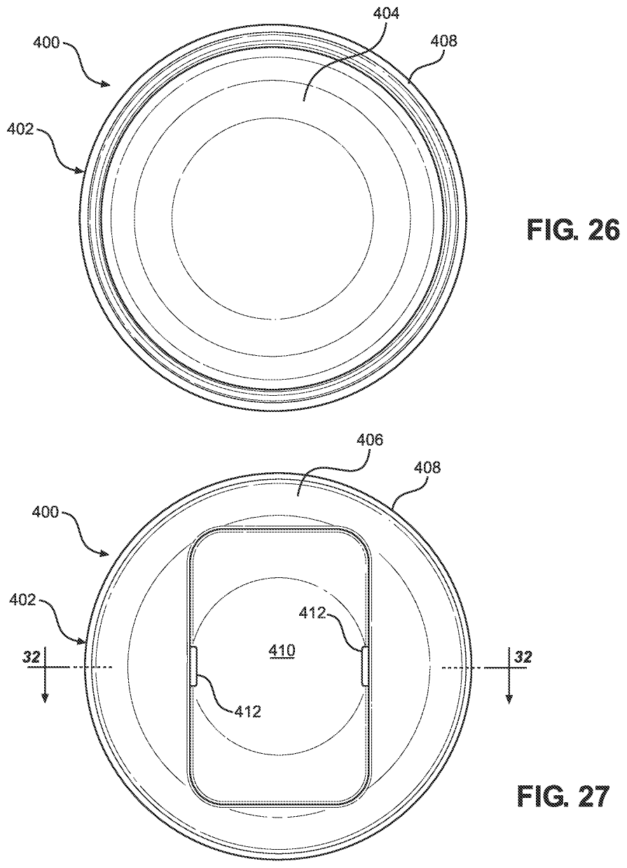

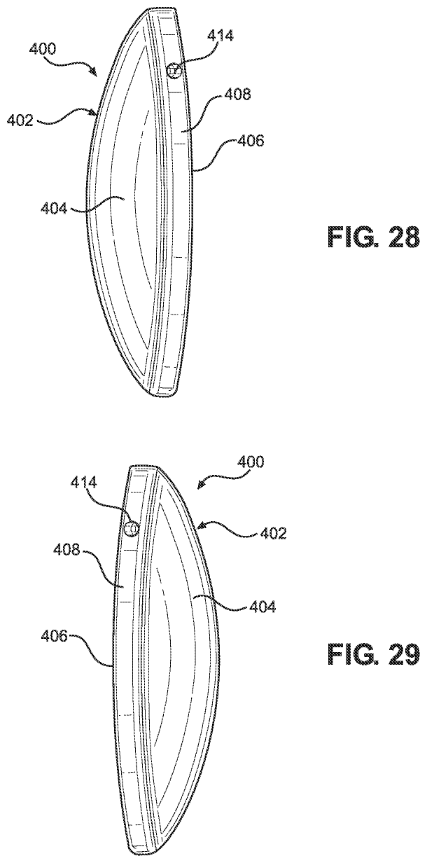

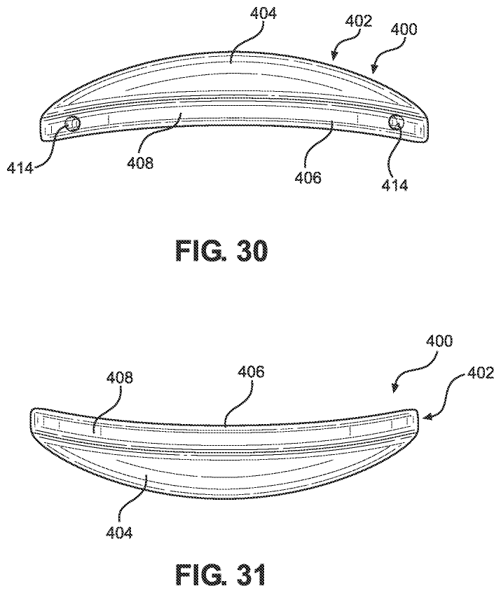

FIGS. 26-31 are posterior, anterior, right side, left side, top, and bottom views, respectively, of the accessory 400. As shown in FIGS. 24-31, the accessory 400 includes a housing 402 having a posterior surface 404, an anterior surface 406, and a circumferential edge 408 extending between the posterior and anterior surfaces 404, 406.

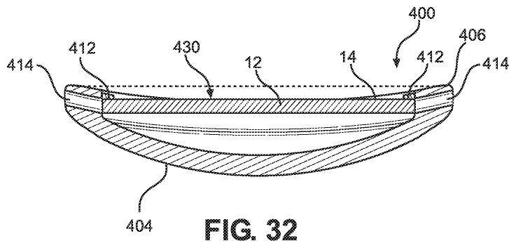

The anterior surface 406 has a cavity 410 shaped and sized to receive an implantable medical device 12 (see FIG. 32). In some examples, the implantable medical device 12 is a conventional spinal cord stimulator device. The cavity 410 is structured to attach the accessory 400 to the implantable medical device 12. When the accessory 400 is attached to the implantable medical device 12, the accessory 400 partially encapsulates the implantable medical device 12. For example, the accessory 400 can cover the posterior, left side, right side, top, and bottom surfaces of the implantable medical device 12 when it is held inside the cavity 410.

In some examples, the housing 402 can be formed of a flexible material such that the housing 402 can flex around the implantable medical device 12, and thereby hold and secure the implantable medical device 12 inside the cavity 410. In some examples, the cavity 410 can include one or more snap fit fasteners 412 that are configured to attach the accessory 400 to the implantable medical device 12. The housing 402 is made of a biocompatible and inert material, such as one or more polymers, including without limitation, silicon rubber, silicone elastomers, polyethylene, polyether ether ketone (PEEK), or any combinations thereof.

FIG. 32 is a cross-sectional view of the accessory 400 taken from the line 32-32 shown in FIG. 27, and with an implantable medical device 12 inside the cavity 410. As shown in FIGS. 24-32, the housing 402 includes one or more bore holes 414 that can receive the leads 10 of the implantable medical device 12. When the implantable medical device 12 is held inside the cavity 410, one or more of the leads 10 can be threaded through the one or more bore holes 414 such that a proximal end of a lead can be attached to the implantable medical device 12, and a distal end of the lead can be implanted in a body area such as the epidural space of the spine.

In the example illustrated in FIGS. 24-32, the accessory 400 includes a bore hole 414 on opposite sides, thereby allowing one or more leads to attach to either side of the implantable medical device 12. In other examples, the accessory 400 includes only one bore hole, or the accessory 400 can include more than two bore holes, as may be desirable.

In the example illustrated in FIGS. 24-32, the posterior surface 404 of the accessory 400 has a convex shape. The convex shape of the posterior surface 404 provides a contour generally conforming to the profile of the second body area where the accessory 400 and implantable medical device 12 are implanted. Advantageously, this can minimize the appearance of the implantable medical device 12 when implanted under the skin of a patient, by mitigating the visual appearance of the defined edges of the implantable medical device.

The convex shape of the posterior surface 404 can vary depending on the desired location for implanting the implantable medical device 12. For example, the convex shape of the posterior surface 404 can be varied to conform to the profile of a buttock, lower back, underarm, chest wall, scalp, and other suitable areas of the body.

As shown in FIGS. 24-32, the circumferential edge 408 is circular. The convex shape of the posterior surface 404 and the circular profile of the circumferential edge 408 provide the housing 402 with a dome-shape. Advantageously, the dome-shape of the housing 402 can help to improve the distribution forces that are applied to the implantable medical device 12 when attached to the accessory 400. For example, when the accessory 400 and implantable medical device 12 are implanted into the buttock of a patient, and the patient sits down, the dome-shape of the housing 402 can help to improve the distribution of the patient's weight applied to the implantable medical device 12 as a result of the patient sitting down.

The convex shape of the posterior surface 404 and the circular profile of the circumferential edge 408 are larger than the surface area and overall size of the implantable medical device 12. This is an unexpected result because implantable medical devices, such as conventional spinal cord stimulators, are designed to be as small as possible.

Referring now to FIG. 32, the accessory 400 is shaped and sized to store the excess slack 26 of one or more of the leads 10 when attached to the implantable medical device 12. Advantageously, storing the excess slack 26 of the one or more leads 10 can reduce pain and discomfort that may be felt by a patient from the implantable medical device 12 and the one or more leads 10 having been implanted into the patient's body. Additionally, the accessory 400 provides a defined storage area for storing the excess slack of the one or more leads 10, which can aid in the removal or replacement of the one or more leads 10.

As shown in FIG. 32, the housing 402 is shaped to define the storage area for storing the excess slack of the one or more leads 10 as a negative space 430 formed by the anterior surface 406 of the accessory 400. The anterior surface 406 surrounds the implantable medical device 12, and the anterior surface 406 has a concave shape that partially defines the negative space 430 for storing the excess slack 26 from the one or more leads 10. Thus, the accessory 400 can store the excess slack 26 of one or more of the leads 10 in a similar fashion as the embodiment of the implantable medical device 100 shown in FIGS. 1-9.

As shown in FIG. 32, the negative space 430 for storing the excess slack is bounded by the anterior surface 406 of the accessory 400 and by an anterior surface 14 of the implantable medical device 12. In the example shown in FIG. 32, the implantable medical device 12 has an anterior surface 14 that is substantially flat. In alternative examples, the negative space 430 can be formed as a cutout or a hollowed portion on the anterior surface 406 of the accessory 400.

In some further examples, the excess slack 26 of the one or more leads 10 can be at least partially coiled around the circumferential edge 408 of the accessory 400 for storage. In such examples, the circular profile of the circumferential edge 408 can help facilitate coiling the excess slack 26 of the one or more leads 10 around the circumferential edge 408. In some examples, the circumferential edge 408 can include a track, groove, or channel for coiling the excess slack 26 of the one or more leads 10 around the circumferential edge 408.

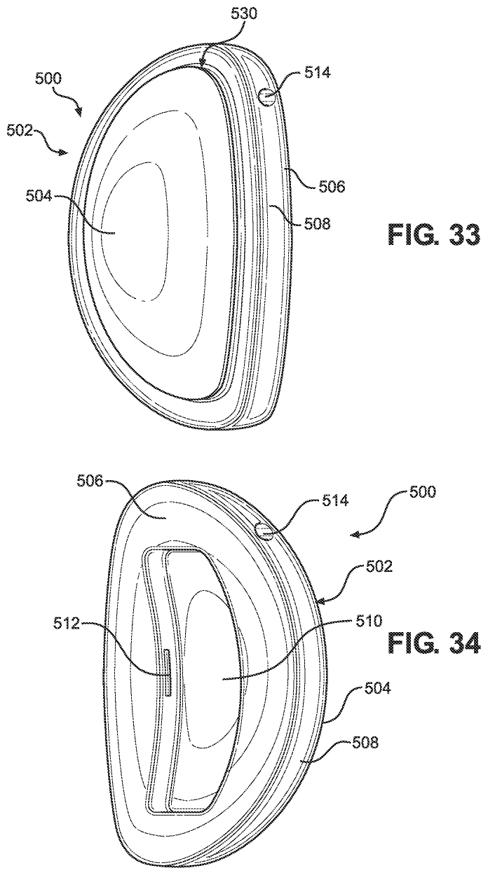

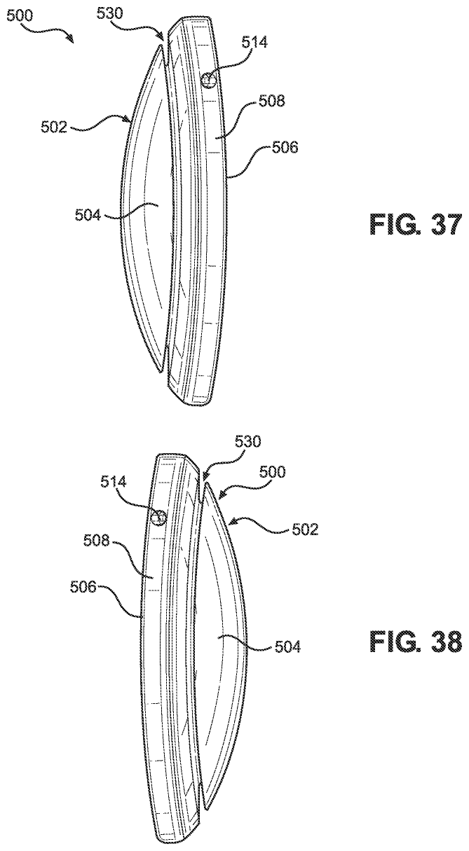

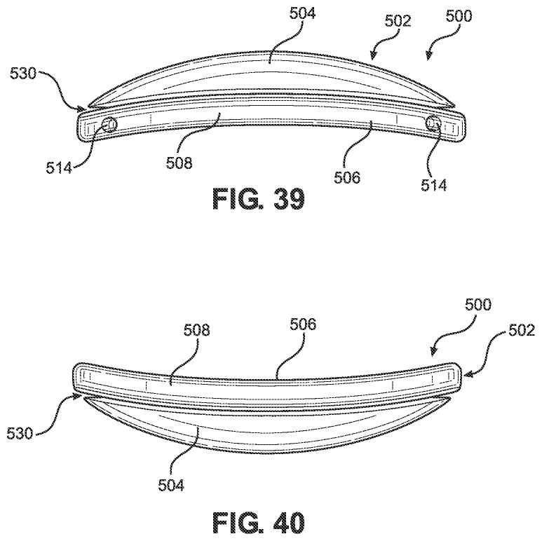

FIGS. 33-41 are isometric posterior, isometric anterior, posterior, anterior, right side, left side, top, bottom, and cross-sectional views, respectively, of an accessory 500 in accordance with another embodiment of the present disclosure. The accessory 500 is shaped and sized to provide a storage area for excess lead slack from an implantable medical device. In a preferred embodiment, the accessory 500 is designed for a conventional spinal cord stimulator device.

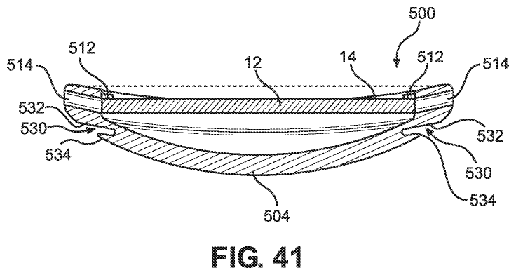

The accessory 500 shares similar elements and features with the accessory 400 described above with respect to FIG. 24-32. For example, the accessory 500 includes a housing 502 having a posterior surface 504, an anterior surface 506, and a circumferential edge 508 extending between the posterior and anterior surfaces 504, 506. The anterior surface 506 has a cavity 510 shaped and sized to receive an implantable medical device 12 (see FIG. 41). In some examples, the implantable medical device 12 is a conventional spinal cord stimulator device.

The cavity 510 is structured to attach the accessory 500 to the implantable medical device 12. When the accessory 500 is attached to the implantable medical device 12, the accessory 500 partially encapsulates the implantable medical device 12. For example, as shown in FIG. 41, the accessory 500 covers the posterior, left side, right side, top, and bottom surfaces of the implantable medical device 12 when it is held inside the cavity 510.

In some examples, the housing 502 can be formed of a flexible material such that the housing 502 can flex around the implantable medical device 12, and thereby hold and secure the implantable medical device 12 inside the cavity 510. In some examples, the cavity 510 can include one or more snap fit fasteners 512 that are configured to attach the accessory 500 to the implantable medical device 12. The housing 502 is made of a biocompatible and inert material, such as one or more polymers, including without limitation, silicon rubber, silicone elastomers, polyethylene, polyether ether ketone (PEEK), or any combinations thereof.

The housing 502 includes one or more bore holes 514 that can receive one or more of the leads 10 that attach to the implantable medical device 12. The one or more bore holes 514 are similar as the bore holes 414 described above with reference to accessory 400.

In the example illustrated in FIGS. 33-41, the posterior surface 504 of the accessory 500 has a convex shape. The convex shape of the posterior surface 504 provides a contour generally conforming to the profile of a location of the body where it is desirable to implant the implantable medical device 12. Advantageously, the convex shape of the posterior surface 504 can minimize the appearance of the implantable medical device 12 when implanted under the skin by mitigating the visual appearance of the defined edges of the implantable medical device.

The convex shape of the posterior surface 504 can vary depending on the desired location for implanting the implantable medical device 12. For example, the convex shape of the posterior surface 504 can be varied to conform the shape of the accessory 500 to the profile of a buttock, lower back, underarm, chest wall, scalp, and other suitable areas of the body.

In the example illustrated in FIGS. 33-41, the circumferential edge 508 is circular. The convex shape of the posterior surface 504 and the circular profile of the circumferential edge 508 provide the housing 502 with a dome-shape that can help to improve the distribution forces applied to the implantable medical device 12 when attached to the accessory 500. For example, when the implantable medical device 12 is implanted into the buttock of a patient, and the patient sits down, the dome-shape of the housing 502 can improve the distribution of the patient's weight applied to the implantable medical device 12 as a result of the patient sitting down.

The convex shape of the posterior surface 504 and the circular profile of the circumferential edge 508 are larger than the surface area and overall size of the implantable medical device 12. This is an unexpected result because implantable medical devices, such as conventional spinal cord stimulators, are designed to be as small as possible.

The housing 502 defines a storage area for storing the excess slack 26 of the one or more leads 10. In the example shown in FIGS. 33-41, the storage area is a track 530 on the posterior surface 504 that allows the one or more leads 10 to be at least partially looped around the housing 502 to reduce the excess slack 26 of the leads. In some examples, one or more of the leads 10 can be looped once, twice, or more around the track 530 for storage. In this example embodiment, another advantage of the circular profile of the circumferential edge 508 is that it facilitates wrapping, winding, or coiling the one or more leads 10 around the track 530.

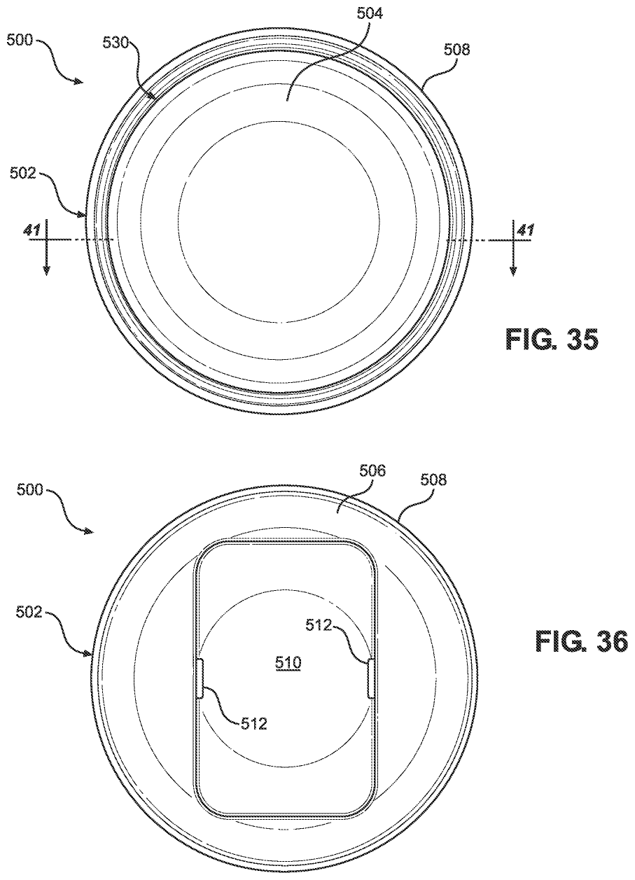

As shown in FIG. 41, which is a cross-sectional view of the accessory 500 taken from the line 41-41 shown in FIG. 35, and which shows the implantable medical device 12 inside the cavity 510, the track 530 includes opposing edges 532, 534 that can catch one or more of the leads 10 for containing the leads inside the track 530. Thus, the accessory 500 can store the excess slack 26 of one or more of the leads 10 in a similar fashion as the embodiment of the implantable medical device 200 shown in FIGS. 10-18. Advantageously, the track 530 can mitigate irritation and discomfort from loose leads, and can minimize the appearance of the excess slack 26 of the one or more leads 10, which may bulge under the skin.

FIG. 42 schematically illustrates components of the implantable medical device 100, 200, 300 in accordance with the embodiments described above. As shown in FIG. 42, the implantable medical device 100, 200, 300 includes at least one processing unit 112 that regulates the operation of the power source 110 to generate the electrical impulses.

The implantable medical device 100, 200, 300 includes a system memory 114. The system memory 114 may include volatile storage (e.g., random access memory), non-volatile storage (e.g., read-only memory), flash memory, or any combinations thereof. The system memory 114 may include an operating system and program modules for running software applications performed by the at least one processing unit 112 to control the operation of the power source 110 and other components of the implantable medical device.

The implantable medical device 100, 200, 300 further includes a communications unit 116 for receiving commands from an external remote control 118. The external remote control 118 allows a patient to adjust the stimulation provided by the implantable medical device 100, 200, 300 such as by regulating the electrical impulses that are generated by the power source 110 and that are released by the electrical contacts of the one or more leads 10.

The implantable medical device 100, 200, 300 includes one or more lead connectors 120 that can receive the proximal ends of the one or more leads 10. As an illustrative example, the implantable medical device 100, 200, 300 can include one, two, three, four, or more lead connectors 120 for connecting one, two, three, four, or more leads to the power source 110.

The implantable medical device 100, 200, 300 includes an electrical circuit 122 that connects the various components illustrated in FIG. 42 including the power source 110, the at least one processing unit 112, the system memory 114, the communications unit 116, and the lead connectors 120. The electrical circuit 122 can be a circuit having discrete electronic elements, packaged or integrated electronic chips containing logic gates, or a single chip containing electronic elements. The electrical circuit 122 can be a system-on-a-chip (SOC) on which the components illustrated in FIG. 42 are integrated. When operating via an SOC, the functionality of the implantable medical device 100, 200, 300 may be operated via application-specific logic integrated with other components on a single integrated circuit.

The various embodiments described above are provided by way of illustration only and should not be construed to be limiting in any way. Various modifications can be made to the embodiments described above without departing from the true spirit and scope of the disclosure.

What is claimed is:

1. An implantable medical device, comprising:
a power source configured to supply electrical impulses to a lead; and
a housing encloses the power source, the housing having:
a posterior surface;
an anterior surface; and
a circumferential edge extending between the posterior and anterior surfaces; and
wherein the housing defines a storage area for excess slack of the lead.

2. The implantable medical device of claim 1, wherein the storage area is a negative space shaped by the anterior surface.

3. The implantable medical device of claim 2, wherein the posterior surface is convex.

4. The implantable medical device of claim 3, wherein the circumferential edge is circular.

5. The implantable medical device of claim 1, wherein the storage area is a track on the circumferential edge.

6. The implantable medical device of claim 5, wherein the track is shaped for looping excess slack of the lead at least partially around the circumferential edge.

7. The implantable medical device of claim 6, wherein the circumferential edge is circular.

8. The implantable medical device of claim 7, wherein the posterior surface is convex.

9. The implantable medical device of claim 1, wherein the storage area is inside the enclosure.

10. The implantable medical device of claim 9, wherein the housing includes a crank for coiling excess slack of the lead for storage inside the enclosure.

11. The implantable medical device of claim 10, wherein the posterior surface is convex.

12. The implantable medical device of claim 1, further comprising:
the lead which extends from a proximal end and a distal end, the proximal end being attachable to the power source, and the distal end having electrical contacts that are configured to deliver the electrical impulses from the power source to a first body area; and
wherein the housing is configured to be implanted in a second body area.

13. The implantable medical device of claim 12, wherein the excess slack of the lead is defined as an excess amount of a first length over a second length, wherein the first length is defined as a distance between the proximal and distal ends of the lead, wherein the second length is defined as a distance between the first and second body areas.

14. The implantable medical device of claim 13, wherein the first body area is an epidural space, and the second body area is a buttock or lower back.

15. An accessory for an implantable medical device, the accessory comprising:
a posterior surface having a convex shape;
an anterior surface having a cavity configured to attach the accessory to a spinal cord stimulator device, and to partially encapsulate the spinal cord stimulator device;
a circumferential edge extending between the posterior and anterior surfaces; and
at least one storage area shaped for storing excess slack of a lead when the lead is connected to the spinal cord stimulator device.

16. The accessory of claim 15, wherein the at least one storage area is a concave surface formed on the anterior surface.

17. The accessory of claim 15, wherein the at least one storage area is a track on the circumferential edge.

18. The accessory of claim 15, further comprising:
a bore extending from the cavity and through the circumferential edge, the bore allowing the lead to reach an epidural space when the lead is connected to the spinal cord stimulator device and the spinal cord stimulator device is attached inside the cavity.

19. The accessory of claim 15, wherein the accessory is made of a flexible material allowing the cavity to flex around the spinal cord stimulator device, and thereby attach the accessory to the spinal cord stimulator device.

20. The accessory of claim 15, wherein the cavity includes one or more snap fit fasteners for attaching the accessory to the spinal cord stimulator device.
